(19) United States
(12) Patent Application Publication
(10) Pub. No.: US 2023/0001042 A1
(43) Pub. Date: Jan. 5, 2023

(54) MEDICAL SUTURE THREAD

(71) Applicant: KEISEI MEDICAL INDUSTRIAL CO., LTD., Tokyo (JP)

(72) Inventor: Jumpei Matsuda, Tokyo (JP)

(21) Appl. No.: 17/782,458

(22) PCT Filed: Nov. 27, 2020
(86) PCT No.: PCT/JP2020/044309

§ 371 Date: Jun. 3, 2022

(30) Foreign Application Priority Data
Dec. 11, 2019 (JP) 2019-223727

(51) Int. Cl. A61L 17/14; A61B 17/06; C07F 9/09; C08F 30/02; C08F 230/02

(52) U.S. Cl. CPC A61L 17/145; A61B 17/06166; C07F 9/091; C08F 30/02; C08F 230/02

(57) ABSTRACT

Provided is a medical suture thread which is less likely to remain curled or is easily uncurled from a curled state. The medical suture thread 100 includes a core thread 110 and an outer thread 120. The core thread 110 includes multiple twisted ultrafine threads 111, and is arranged at a center portion of the medical suture thread 100. In the ultrafine thread 111, an inner-filament cover layer 112 made of 2-methacryloyloxyethyl phosphorylcholine (MPC) is formed on an outer surface of a filament 111a. The outer thread 120 is formed to be braided with multiple ultrafine threads 121, and covers an outer surface of the core thread 110. In the ultrafine thread 121, an outer-filament cover layer 122 made of MPC is formed on an outer surface of a filament 121a. The inner-filament cover layer 112 and the outer-filament cover layer 122 are respectively formed on the outer surfaces of the filaments 111a, 121a within a weight range of equal to or greater than 0.05% and less than 0.3% with respect to the total weight of each of the filaments 111a, 121a, respectively.

### TECHNICAL FIELD

The present invention relates to a medical suture thread used for medical action such as a surgery or injury treatment.

### BACKGROUND ART

Typically, a medical suture thread used for medical action such as a surgery or injury treatment has been used. Of this medical suture thread, an outer surface is coated with various resin materials for the purpose of improving operability in suturing and slidability or slide-down performance (the easiness of moving a knot upon thread tying) on a sutured portion. For example, Patent Literature 1 below discloses that a suture thread is coated with 2-methacryloyloxyethyl phosphorylcholine (hereinafter referred to as “MPC”). This can enhance easiness and retainability when a surgeon ties the suture thread.

### CITATION LIST

### Patent Literature

PATENT LITERATURE 1: JP-A-2010-513678

However, there has been a problem that the suture thread described in Patent Literature 1 above is likely to remain curled after the suture thread housed in a packaging container with wound in a circular ring shape is taken out of the packaging container. In addition, there has also been a problem that it is difficult to uncurl the suture thread from a curled state.

The present invention has been made to cope with the above-described problems. An object of the present invention is to provide a medical suture thread which is less likely to remain curled or is easily uncurled from a curled state.

### SUMMARY OF INVENTION

In order to achieve the above object, as a feature of the present invention, there is provided a medical suture thread including a single thread having one or a plurality of fibrous ultrafine threads. A cover layer made of 2-methacryloyloxyethyl phosphorylcholine (MPC) is formed on an outer surface of each ultrafine thread, and the cover layer is formed to have a weight of equal to or greater than 0.05% and less than 0.3% with respect to a total weight of a filament forming each ultrafine thread, the cover layer being formed on the filament.

According to the feature of the present invention configured as described above, in the medical suture thread, the cover layer made of 2-methacryloyloxyethyl phosphorylcholine (MPC) is formed with a weight of equal to or greater than 0.05% and less than 0.3% with respect to the total weight of the filament forming the ultrafine thread. With this configuration, according to experiment conducted by the present inventor, operability in suturing and slidability or slide-down performance on a sutured portion are improved. In addition, it has been confirmed that the medical suture thread housed in a circular ring shape in a packaging container is less likely to remain curled or is easily uncurled from a curled state.

Moreover, as another feature of the present invention, in the medical suture thread, the cover layer is formed to have a weight of equal to or greater than 0.1% and less than 0.3% with respect to a total weight of a filament forming each ultrafine thread, the cover layer being formed on the filament.

According to another feature of the present invention configured as described above, in the medical suture thread, the cover layer is formed with a weight of equal to or greater than 0.1% and less than 0.3% with respect to the total weight of the filament forming the ultrafine thread. Thus, the medical suture thread can be easily formed as compared to the case of forming the cover layer with a weight of less than 0.1% with respect to the total weight of the filament.

Moreover, as another feature of the present invention, in the medical suture thread, the cover layer is formed such that part of an outer surface of the filament is exposed.

According to another feature of the present invention configured as described above, in the medical suture thread, the cover layer is formed such that part of the outer surface of the filament is exposed. Thus, the cover layer is not necessarily formed across the entirety of the outer surface of the filament. Consequently, the amount of MPC to be used and a burden in the process of forming the cover layer can be reduced. Accordingly, the cover layer can be efficiently formed.

Moreover, as another feature of the present invention, in the medical suture thread, the cover layer is formed to cover 50% or more of a surface area of the filament with a uniform formation density in each of an axial direction and a peripheral direction of the filament.

According to another feature of the present invention configured as described above, in the medical suture thread, the cover layer is formed to cover 50% or more of the surface area of the filament with the uniform formation density in each of an axial direction and a peripheral direction of the filament. Thus, the cover layer made of MPC can maintain various characteristics such as the operability, the slidability, and the slide-down performance while the amount of MPC to be used and the burden in the process of forming the cover layer can be reduced. Consequently, the cover layer can be efficiently formed.

### BRIEF DESCRIPTION OF DRAWINGS

FIG. 1 is an exploded side view schematically showing the outline of an external configuration and an internal configuration of a medical suture thread according to one embodiment of the present invention;

FIG. 2 is an exploded side view schematically showing the outline of an external configuration and an internal configuration of an ultrafine thread forming each of a core thread and an outer thread in the medical suture thread shown in FIG. 1; and

FIG. 3 is a partially-enlarged side view showing the state of a cover layer partially formed on an outer surface of an ultrafine thread forming a medical suture thread according to a variation of the present invention.

### DESCRIPTION OF EMBODIMENTS

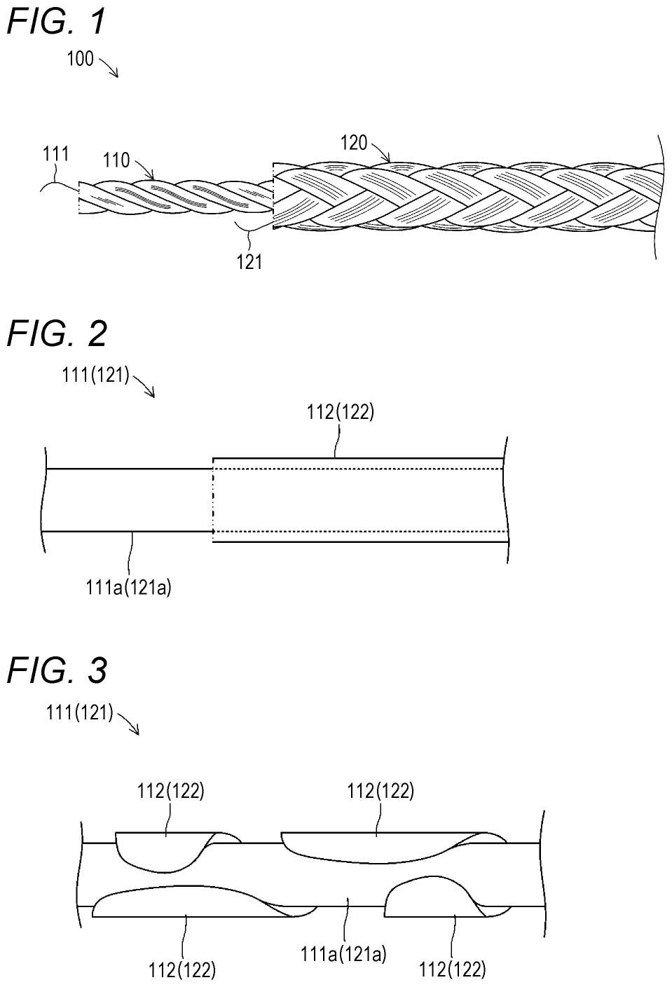

Hereinafter, one embodiment of a medical suture thread according to the present invention will be described with reference to the drawings. FIG. 1 is an exploded side view schematically showing the outline of an external configuration and an internal configuration of a medical suture thread 100 according to the present invention. Note that in each figure as a reference in the present specification, some components are schematically shown using, e.g., exaggerated notation for the sake of easy understanding of the present invention. Thus, the dimensions of the components, the ratios among the components, and the like may vary. The medical suture thread 100 is a tool for suturing part of body tissues such as the skin, muscles, or organs of a human or an animal.

(Configuration of Medical Suture Thread 100)

The medical suture thread 100 mainly includes a core thread 110 and an outer thread 120. The core thread 110 is a thread arranged inside the medical suture thread 100. The core thread 110 includes an assembly of multiple ultrafine threads 111. In the present embodiment, two bundles of multiple assembled ultrafine threads 111 are twisted to form a single thread in the configuration of the core thread 110. Thus, the core thread 110 is, in the present embodiment, formed to have a thickness of about 0.27 mm.

As shown in FIG. 2, the ultrafine thread 111 is the minimum unit of thread forming the core thread 110. The ultrafine thread 111 includes a filament 111a made of a resin material, a natural material, or a metal material. In this case, the resin material to be used may include, for example, nylon, polypropylene, polyester, and polyvinylidene fluoride (PVDF). The natural material to be used may include catgut (a thread formed of twisted animal intestines) and a silken thread. The metal material to be used may include stainless steel, titanium, and magnesium.

The thickness of the ultrafine thread 111 varies according to the thickness of the medical suture thread 100. The ultrafine thread 111 is formed such that the thickness thereof is equal to or greater than about 1 μm and equal to or less than about 1 mm. In the present embodiment, the ultrafine thread 111 is formed of a polyester thread with a thickness of about 10 μm.

In the ultrafine thread 111, an inner-filament cover layer 112 is formed on a surface of the filament 111a. The inner-filament cover layer 112 is a resin layer covering the outer surface of the filament 111a of the ultrafine thread 111 and containing 2-methacryloyloxyethyl phosphorylcholine (MPC). In this case, the inner-filament cover layer 112 may be made only of MPC. Alternatively, the inner-filament cover layer 112 may be formed such that MPC is contained as a main component, of which content rate is the highest as compared to the other components, among the components forming the inner-filament cover layer 112. In the present embodiment, the inner-filament cover layer 112 is made only of MPC.

The inner-filament cover layer 112 is preferably formed to completely cover the outer surface of the filament 111a. The inner-filament cover layer 112 may be formed to cover 50% or more (more preferably 80% or more) of the surface area of the filament 111a with the substantially uniform formation density in each of axial and peripheral directions of the filament 111a. That is, the inner-filament cover layer 112 may be, as shown in FIG. 3, formed on the outer surface of the filament 111a in a patchy manner with part of the outer surface being exposed.

The substantially uniform formation density in each of the axial and peripheral directions of the filament 111a as described herein indicates a state in which a portion (a portion formed with no inner-filament cover layer 112) with the exposed outer surface of the filament 111a and a portion formed with the inner-filament cover layer 112 are substantially equally arranged in each of the axial and peripheral directions of the filament 111a. It may only be required that when the outer surface of the filament 111a is observed using a magnifying glass such as an electronic microscope, the formation density is substantially uniform to such an extent that the exposed portion of the outer surface of the filament 111a and the portion of the outer surface formed with the inner-filament cover layer 112 seem to be equally arranged. Such arrangement is not necessarily precisely-equal arrangement.

The inner-filament cover layer 112 is formed on the outer surface of the filament 111a within a weight range of equal to or greater than 0.05% and less than 0.3% with respect to the total weight of the filament 111a. That is, the inner-filament cover layer 112 is formed on the outer surface of the filament 111a within a weight range of equal to or greater than 0.05% and less than 0.3% with respect to the total weight of the filament 111a. This can reduce hardening of the medical suture thread 100. Moreover, flexibility can be held. In this case, the thickness of the inner-filament cover layer 112 is set according to use application of the medical suture thread 100 or a user's preference. In the present embodiment, the inner-filament cover layer 112 is formed on the outer surface of the filament 111a with a weight of 0.1% with respect to the total weight of the filament 111a.

The outer thread 120 is a thread arranged to cover an outer surface of the above-described core thread 110. The outer thread 120 includes an assembly of multiple ultrafine threads 121. In the present embodiment, the outer thread 120 is formed in such a manner that six bundles of multiple assembled ultrafine threads 121 are braided to form a single thread. Thus, the outer thread 120 is formed with a thickness of about 0.6 mm in the present embodiment. That is, in the present embodiment, the medical suture thread 100 is formed with a thickness of about 0.6 mm. The outer thread 120 is formed to completely cover the outer surface of the core thread 110.

The ultrafine thread 121 is the minimum unit of thread forming the outer thread 120. The ultrafine thread 121 includes a filament 121a made of a resin material, a natural material, or a metal material. In this case, the resin material to be used may include, for example, nylon, polypropylene, polyester, and polyvinylidene fluoride (PVDF). The natural material to be used may include catgut (a thread formed of twisted animal intestines) and a silken thread. The metal material to be used may include stainless steel, titanium, and magnesium.

The thickness of the ultrafine thread 121 varies according to the thickness of the medical suture thread 100. The ultrafine thread 121 is formed such that the thickness thereof is equal to or greater than about 1 μm and equal to or less than about 1 mm. In the present embodiment, the ultrafine thread 121 is formed of a polyester thread with a thickness of about 10 μm. That is, the ultrafine threads 121 is formed of the exact same thread as that of the ultrafine thread 111 in the present embodiment. Note that the ultrafine thread 121 is formed as necessary according to the specifications of the medical suture thread 100. Thus, the ultrafine thread 121 may be made of a material different from that of the ultrafine thread 111, and/or may be formed with a diameter different from that of the ultrafine thread 111, needless to say.

As shown in FIG. 2, in the ultrafine thread 121, an outer-filament cover layer 122 is formed on a surface of the filament 121a. The outer-filament cover layer 122 is a resin layer covering the outer surface of the filament 121a and containing 2-methacryloyloxyethyl phosphorylcholine (MPC). In this case, the outer-filament cover layer 122 may be made only of MPC. Alternatively, the outer-filament cover layer 122 may be formed such that MPC is contained as a main component, of which content rate is the highest as compared to the other components, among the components forming the outer-filament cover layer 122. The outer-filament cover layer 122 may be made of a resin material other than MPC or synthetic resin containing, as a main component, resin other than MPC. In the present embodiment, the outer-filament cover layer 122 is made only of MPC.

The outer-filament cover layer 122 is preferably formed to completely cover the outer surface of the filament 121a. The outer-filament cover layer 122 may be formed to cover 50% or more (more preferably 80% or more) of the surface area of the ultrafine thread 121 with a substantially uniform formation density in each of axial and peripheral directions of the filament 121a. That is, the outer-filament cover layer 122 may be, as shown in FIG. 3, formed on the outer surface of the filament 121a in a patchy manner with part of the outer surface being exposed. Note that the substantially uniform formation density in each of the axial and peripheral directions of the filament 121a as described herein is similar to that of the inner-filament cover layer 112. Thus, description thereof will be omitted.

The outer-filament cover layer 122 is formed on the outer surface of the filament 121a within a weight range of equal to or greater than 0.05% and less than 0.3% with respect to the total weight of the filament 121a. That is, the outer-filament cover layer 122 is formed on the outer surface of the filament 121a within a weight range of equal to or greater than 0.05% and less than 0.3% with respect to the total weight of the filament 121a. This can provide the flexibility or smooth texture of the medical suture thread 100. In this case, the thickness of the outer-filament cover layer 122 is set according to use application of the medical suture thread 100 or a user's preference. In the present embodiment, the outer-filament cover layer 122 is, as in the inner-filament cover layer 112, formed on the outer surface of the filament 121a with a weight of 0.1% with respect to the total weight of the filament 121a.

That is, the ultrafine thread 121 has the exact same configuration as that of the ultrafine thread 111 in the present embodiment. Note that the outer-filament cover layer 122 with a configuration different from that of the inner-filament cover layer 112 may be formed. Alternatively, the ultrafine thread 121 may be formed without the outer-filament cover layer 122. Note that FIG. 1 shows one extracted ultrafine thread 111 and one extracted ultrafine thread 121 for the sake of easy understanding of the internal structure of the medical suture thread 100. In addition, the inner-filament cover layer 112 and the outer-filament cover layer 122 are not shown in the figure. Each of FIGS. 2 and 3 shows, by one common view, the ultrafine thread 111 and the ultrafine thread 121.

(Manufacturing of Medical Suture Thread 100)

Next, the process of manufacturing the medical suture thread 100 will be described. A worker manufacturing the medical suture thread 100 first prepares the ultrafine thread 111 formed with no inner-filament cover layer 112 and the ultrafine thread 121 formed with no outer-filament cover layer 122. Specifically, the filaments 111a, 121a are prepared as bases of the ultrafine threads 111, 121.

In this case, the filaments 111a, 121a are manufactured by the typical technique of manufacturing a suture thread. Thus, the worker may manufacture the filaments 111a, 121a as the bases of the ultrafine threads 111, 121 by the worker oneself. Alternatively, a commercially-available filament may be purchased and obtained. In the present embodiment, the worker prepares the polyester filaments 111a, 121a with a size of No. 11-0 (about 20 μm) (the standard for a suture thread according to the Pharmaceutical Affairs Act).

Next, the worker forms the inner-filament cover layer 112 and the outer-filament cover layer 122 on the filaments 111a, 121a. Specifically, the worker dips the filaments 111a, 121a in a solution (e.g., an ethanol solution) containing MPC. In this manner, the inner-filament cover layer 112 and the outer-filament cover layer 122 can be formed on the surfaces of the filaments 111a, 121a.

In this case, the worker can dip the filament in the solution containing MPC in a state in which the multiple filaments 111a, 121a are gathered and bundled, a roll state in which each of the filaments 111a, 121a is wound in an annular shape, or a state in which each of the filaments 111a, 121a wound in a roll shape is drawn in a linear shape. The concentration of the solution containing MPC and the time of dipping the filaments 111a, 121a are respectively determined as necessary according to the thicknesses of the inner-filament cover layer 112 and the outer-filament cover layer 122 to be formed.

Then, the worker performs drying treatment and sterilization treatment for the filaments 111a, 121a taken out of the solution containing MPC. In this manner, the worker can obtain the ultrafine threads 111, 121 formed with the inner-filament cover layer 112 and the outer-filament cover layer 122 on the outer surfaces of the filaments 111a, 121a, respectively. Note that the worker provides vibration to the filaments 111a, 121a or sets a drying temperature to a high temperature upon drying of the filaments 111a, 121a, and in this manner, can form the inner-filament cover layer 112 and the outer-filament cover layer 122 on the surfaces of the filaments 111a, 121a in a patchy manner.

Next, the worker forms the core thread 110. Specifically, the worker twists two bundles of multiple gathered ultrafine threads 111 by means of a not-shown twisting machine. In this manner, the single core thread 110 is formed. In the present embodiment, the worker can form the core thread 110 with a thickness of about 0.27 mm.

Next, the worker forms the outer thread 120. Specifically, the worker braids the outer thread 120, which is formed of five bundles of multiple gathered ultrafine threads 121, on the outside of the core thread 110 by means of a not-shown braiding machine. In this manner, a single thread is formed. That is, by the step of forming the outer thread 120, the medical suture thread 100 including the core thread 110 completely covered with the outer thread 120 is formed. The medical suture thread 100 is formed with an outer diameter of about 0.6 mm in the present embodiment.

Next, the worker can complete the medical suture thread 100 through the steps of sterilizing, checking, and packing the medical suture thread 100. In this case, the medical suture thread 100 is housed in a packaging container (not shown) in a state in which the medical suture thread 100 is wound in a circular ring shape. Alternatively, the medical suture thread 100 may be packed with a medical tool such as a suture needle being coupled to a tip end portion of the medical suture thread 100. Note that the worker can form the inner-filament cover layer 112 and the outer-filament cover layer 122 on a medical suture thread 100 formed with no inner-filament cover layer 112 and no outer-filament cover layer 122.

Specifically, the worker processes the filaments 111a by means of the not-shown twisting machine. After formation of the core thread 110, the ultrafine threads 121 are braided using the not-shown braiding machine and the filaments 121a. In this manner, the medical suture thread 100 (i.e., a half-finished product of the medical suture thread 100) is formed at an outer peripheral portion of the core thread 110. Next, the worker dips the half-finished product of the medical suture thread 100 in the solution containing MPC. In this case, the worker causes a sufficient solution to penetrate the medical suture thread 100 to each ultrafine thread 111 (each filament 111a) forming the core thread 110.

In this case, the worker can intermittently change tension to be applied to the half-finished product of the medical suture thread 100 in the solution containing MPC. In this manner, a sufficient solution can penetrate the medical suture thread 100 to each ultrafine thread 111 (each filament 111a) forming the core thread 110. In this manner, the worker forms the inner-filament cover layer 112 and the outer-filament cover layer 122 on the outer surfaces of the filaments 111a, 121a, respectively. Accordingly, the worker can obtain the medical suture thread 100 formed of the ultrafine threads 111, 121 including these filaments and cover layers.

(Operation of Medical Suture Thread 100)

Next, operation of the medical suture thread 100 configured as described above will be described. A user such as a doctor couples a medical tool (not shown) such as a suture needle to the medical suture thread 100 unpacked from the package. Then, the user takes medical action such as a surgery or injury treatment. In this case, in the medical suture thread 100, the inner-filament cover layer 112 formed on the ultrafine thread 111 is formed with a weight of 0.1% with respect to the total weight of the filament 111a. Moreover, the outer-filament cover layer 122 formed on the ultrafine thread 121 is formed with a weight of 0.1% with respect to the total weight of the filament 121a. Thus, the medical suture thread 100 is less likely to remain curled. In addition, even in a case where the medical suture thread 100 remains curled, the medical suture thread 100 is uncurled from a curled state within a short period of time, and extends straight. In this case, in a state in which the medical suture thread 100 is wound in the circular ring shape, adjacent turns of the medical suture thread 100 are less likely to adhere to each other. Thus, the user can easily extend the medical suture thread 100 straight.

Moreover, the user takes medical action such as removal of the medical suture thread 100 sutured to an affected area of a patient. In this case, in the medical suture thread 100, not only the outer thread 120 but also the core thread 110 inside the outer thread 120 are arranged, and the inner-filament cover layer 112 made of MPC is formed as an outer layer of each ultrafine thread 111 forming the core thread 110. Thus, friction among the ultrafine threads 111 is reduced. Consequently, the medical suture thread 100 is easily bendable. As a result, the user can easily operate the medical suture thread 100.

As can be understood from description of operation above, according to the above-described embodiment, in the medical suture thread 100, a cover layer including the inner-filament cover layer 112 made of MPC is formed with a weight of equal to or greater than 0.05% and less than 0.3% with respect to the total weight of the filament 111a forming the ultrafine thread 111. Moreover, a cover layer including the outer-filament cover layer 122 made of MPC is formed with a weight of equal to or greater than 0.05% and less than 0.3% with respect to the total weight of the filament 121a forming the ultrafine thread 121. With this configuration, the medical suture thread 100 housed in the circular ring shape in the packaging container is less likely to remain curled, or can be easily uncurled from the curled state.

Further, the embodiment of the present invention is not limited to the above-described embodiment. Various changes can be made to the above-described embodiment without departing from the object of the present invention.

For example, in the above-described embodiment, the inner-filament cover layer 112 and the outer-filament cover layer 122 are formed with a weight of 0.1% with respect to the total weight of each of the filaments 111a, 121a, respectively. That is, the inner-filament cover layer 112 and the outer-filament cover layer 122 are equivalent to the cover layer according to the present invention. However, according to experiment conducted by the present inventor, it may only be required that the inner-filament cover layer 112 and the outer-filament cover layer 122 are formed within a weight range of equal to or greater than 0.05% and less than 0.3% with respect to the total weight of each of the filaments 111a, 121a, respectively. In this case, the inner-filament cover layer 112 and the outer-filament cover layer 122 are formed within a weight range of equal to or greater than 0.1% and less than 0.3% with respect to the total weight of each of the filaments 111a, 121a, respectively. With this configuration, the inner-filament cover layer 112 and the outer-filament cover layer 122 can easily form the cover layer as compared to the case of forming such a layer with a weight of less than 0.1% with respect to the total weight of each of the filaments 111a, 121a.

In the above-described embodiment, the inner-filament cover layer 112 and the outer-filament cover layer 122 are formed as a coating completely covering the outer surfaces of the filaments 111a, 121a, respectively. However, the inner-filament cover layer 112 and the outer-filament cover layer 122 may be respectively formed such that part of the outer surface of each of the filaments 111a, 121a is exposed as shown in FIG. 3. With this configuration, in the medical suture thread 100, the inner-filament cover layer 112 or the outer-filament cover layer 122 is not necessarily formed across the entirety of the outer surface of the filament 111a or 121a. Thus, the amount of MPC to be used and a burden in the process of forming the inner-filament cover layer 112 and the outer-filament cover layer 122 can be reduced. Accordingly, the inner-filament cover layer 112 and the outer-filament cover layer 122 can be efficiently formed.

In the above-described embodiment, in the medical suture thread 100, the inner-filament cover layer 112 is formed on the ultrafine thread 111 forming the core thread 110. Moreover, the outer-filament cover layer 122 is formed on the ultrafine thread 121 forming the outer thread 120. However, it may only be required that in the configuration of the medical suture thread 100, the inner-filament cover layer 112 or the outer-filament cover layer 122 is formed on the ultrafine thread 111 or the ultrafine thread 121 forming one of the core thread 110 or the outer thread 120.

In this case, in the medical suture thread 100, the outer-filament cover layer 122 can be formed only on the outer thread 120 or the outer thread 120 exposed at an outer surface of the medical suture thread 100. With this configuration, operability in suturing and slidability or slide-down performance on a sutured portion can be improved. In addition, the medical suture thread 100 is less likely to remain curled, or can be easily uncurled from the curled state.

In the above-described embodiment, in the medical suture thread 100, the inner-filament cover layer 112 of the ultrafine thread 111 forming the core thread 110 and the outer-filament cover layer 122 of the ultrafine thread 121 forming the outer thread 120 are formed with the same thickness (a weight of 0.1% with respect to the total weight of each of the ultrafine threads 111, 121). However, the inner-filament cover layer 112 and the outer-filament cover layer 122 may be formed with different thicknesses.

Thus, the inner-filament cover layer 112 may be formed thinner than the outer-filament cover layer 122, for example. With this configuration, an increase in the outer diameter of the medical suture thread 100 can be suppressed while the bendability of the entirety of the medical suture thread 100 is improved. Thus, favorable operability can be ensured. The inner-filament cover layer 112 may be formed thicker than the outer-filament cover layer 122. With this configuration, a loss of the inner-filament cover layer 112 due to abrasion can be reduced. Moreover, the bendability can be maintained over a long period of time.

In the above-described embodiment, the core thread 110 includes the multiple twisted ultrafine threads 111. Specifically, in the configuration of the core thread 110, two bundles of multiple gathered ultrafine threads 111 are twisted. However, in the configuration of the core thread 110, three or more bundles may be twisted. The core thread 110 may be formed in such a manner that multiple bundles of multiple ultrafine threads 111 are braided. In addition, a configuration including multiple ultrafine threads 111 linearly arranged in parallel may be employed. The core thread 110 may include a single ultrafine thread 111.

In the above-described embodiment, the outer thread 120 is formed in such a manner that the multiple ultrafine threads 121 are braided. Specifically, the outer thread 120 is formed in such a manner that five bundles of multiple gathered ultrafine threads 121 are braided. However, the outer thread 120 may be formed in such a manner that four or less or six or more bundles are braided. The outer thread 120 may include multiple twisted bundles of multiple ultrafine threads 121.

In the above-described embodiment, in the configuration of the medical suture thread 100, the outer thread 120 is formed outside the core thread 110. However, it may only be required that the medical suture thread 100 includes a single thread having an assembly of one or multiple fibrous ultrafine threads. Thus, the medical suture thread 100 may include only one ultrafine thread 111 or one ultrafine thread 121.

### LIST OF REFERENCE SIGNS

100 Medical Suture Thread
110 Core Thread
111 Ultrafine Thread
111a Filament
112 Inner-Filament Cover Layer
120 Outer Thread
121 Ultrafine Thread
121a Filament
122 Outer-Filament Cover Layer

1. A medical suture thread comprising:
a single thread having one or a plurality of fibrous ultrafine threads,
wherein a cover layer made of 2-methacryloyloxyethyl phosphorylcholine (MPC) is formed on an outer surface of each ultrafine thread, and
the cover layer is formed to have a weight of equal to or greater than 0.05% and less than 0.3% with respect to a total weight of a filament forming each ultrafine thread, the cover layer being formed on the filament.

2. The medical suture thread according to claim 1, wherein
the cover layer is formed to have a weight of equal to or greater than 0.1% and less than 0.3% with respect to the total weight of the filament forming each ultrafine thread, the cover layer being formed on the filament.

3. The medical suture thread according to claim 1, wherein
the cover layer is formed such that part of an outer surface of the filament is exposed.

4. The medical suture thread according to claim 3, wherein
the cover layer is formed to cover 50% or more of a surface area of the filament with a uniform formation density in each of an axial direction and a peripheral direction of the filament.
